(19) United States
(12) Patent Application Publication
(10) Pub. No.: US 2021/0162159 A1
(43) Pub. Date: Jun. 3, 2021

(54) SCENT INFUSED NASAL CANNULA AND METHODS OF USE AND FABRICATION THEREOF

(71) Applicant: First Street Concepts, LLC, Springfield, OH (US)

(72) Inventors: Patrick Michael O'Neill, Springfield, OH (US); Robert Alexander Armentrout, Englewood, OH (US); Salim Oblen Dahdah, Beavercreek, OH (US)

(73) Assignee: First Street Concepts, LLC, Springfield, OH (US)

(21) Appl. No.: 17/015,654

(22) Filed: Sep. 9, 2020

Related U.S. Application Data
(63) Continuation of application No. 15/328,545, filed on Jan. 24, 2017, now abandoned.
(60) Provisional application No. 62/028,372, filed on Jul. 24, 2014.

(51) Int. Cl. A61M 16/06; A61M 16/08

(52) U.S. Cl. CPC A61M 16/0672; A61M 15/08; A61M 16/0875

(57) ABSTRACT

Scent infused nasal cannulas, scent infused nasal cannula assemblies, methods of providing scents to subjects, and methods of fabricating scent infused nasal cannulas. The scent infused nasal cannulas and scent infused nasal cannula assemblies include a tubular body, a pair of narine projections, and fluid supply tubes. The tubular body and the pair of narine projections are formed from a scent infused polymer. The methods of providing scents to subjects include attaching a scent infused nasal cannula to a subject and delivering a fluid from a fluid source to the subject during the emission period of the scent infused polymer such that the scent is detectable therefrom. The methods of fabricating scent infused nasal cannulas include providing a scent infused nose piece and attaching fluid supply tubes to the nose piece and to a fluid source.

### CROSS-REFERENCES TO RELATED APPLICATIONS

This application is a continuation of U.S. patent application Ser. No. 15/328,545, filed Jan. 24, 2017, which is a national-stage application under 35 U.S.C. § 371 of International Application No. PCT/US2015/041968, filed Jul. 24, 2015, which International Application claims the benefit of priority to U.S. Provisional Patent Application No. 62/028,372, filed Jul. 24, 2014, the contents of which are hereby incorporated by reference in their entirety.

### TECHNICAL FIELD

The present disclosure relates generally to nasal cannulas. More particularly, the present disclosure relates to one or more of scent infused nasal cannulas, scent infused nasal cannula assemblies, and to methods of use and fabrication thereof.

### BACKGROUND

Nasal cannulas are generally employed to deliver fluids, e.g., supplemental oxygen and/or aerosol therapeutics, to a patient. A typical nasal cannula includes tubing with prongs which are placed in the nostrils of the patient and from which the fluids are delivered. However, the use of such nasal cannulas can be uncomfortable to the patient, leading to an uncomfortable, unpleasant, and/or stressful experience to the patient wearing the nasal cannula. Accordingly, additional embodiments of nasal cannulas are desired.

### SUMMARY

In some embodiments, scent infused nasal cannulas for nasal delivery of nasal fluids from fluid sources to subjects are disclosed. Such cannulas include a tubular body defining a first fluid inlet and a second fluid inlet, a pair of narine projections defining a first fluid outlet and a second fluid outlet, a first fluid supply tube, and a second fluid supply tube. The pair of narine projections are in fluidic communication with the tubular body and are integral with and outwardly extending from the tubular body. The tubular body and the pair of narine projections are formed from a scent infused polymer from which a scent emits for an emission period. The first fluid supply tube is attachable to the first inlet of the tubular body and is attachable to the fluid source. The second fluid supply tube is attachable to the second inlet of the tubular body and is attachable to the fluid source. When the first fluid supply tube and the second fluid supply tube are attached to the first inlet of the tubular body, to the second inlet of the tubular body, and to the fluid source, the first fluid supply tube and the second fluid supply tube are in fluidic communication with the tubular body and with the fluid source for delivery of the fluid from the fluid source to the subject.

In other embodiments, scent infused nasal cannula assemblies for nasal delivery of fluids from fluid sources to subjects are disclosed. Such assemblies include a tubular body defining a first fluid inlet and a second fluid inlet, a pair of narine projections defining a first fluid outlet and a second fluid outlet, a first fluid supply tube including a first supply inlet and a first supply outlet, a second fluid supply tube including a second supply inlet and a second supply outlet, and a third fluid supply tube attached to the supply source and including a third supply outlet. The pair of narine projections are in fluidic communication with the tubular body and are integral with and outwardly extending from the tubular body. The tubular body and the pair of narine projections are formed from a scent infused polymer from which a scent emits for an emission period. The first supply outlet is attached to the first fluid inlet of the tubular body, the second supply outlet is attached to the second fluid inlet of the tubular body, and the third supply outlet is attached to the first supply inlet and the second supply inlet.

In yet other embodiments, methods for providing scents to subjects during nasal delivery of fluids from fluid sources to the subjects are disclosed. The methods include attaching a scent infused nasal cannula to the subject and delivering the fluid from the fluid source to the subject via the scent infused nasal cannula. The scent infused nasal cannula includes a nose piece and a pair of fluid supply tubes in fluidic communication with the nose piece and in fluidic communication with the fluid source. The nose piece is formed from a scent infused polymer from which a scent emits for an emission period. The fluid is delivered during at least a portion of the emission period of the scent infused polymer such that the scent is detectable therefrom.

In still other embodiments, methods of fabricating scent infused nasal cannulas for nasal delivery of fluids from fluid sources to subjects are disclosed. Such methods include providing a scent infused nose piece including a tubular body and a pair of narine projections integral with and in fluidic communication with the tubular body and attaching a pair of fluid supply tubes to the first inlet of the tubular body, to the second inlet of the tubular body, and to the fluid source. The tubular body defines a first fluid inlet and a second fluid inlet. The pair of narine projections define a first fluid outlet and a second fluid outlet. The scent infused nose piece is formed from a scent infused polymer from which a scent emits for an emission period.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 is a frontal view of a scent infused nasal cannula positioned on a patient; and

FIG. 2 is a rear view of the scent infused nasal cannula nose piece of FIG. 1.

Skilled artisans appreciate that elements in the figures are illustrated for simplicity and clarity and are not necessarily drawn to scale. For example, the dimensions of some of the elements in the figures may be exaggerated relative to other elements, as well as conventional parts removed, to help to improve understanding of the various embodiments of the present disclosure.

### DETAILED DESCRIPTION

The following terms are used in the present application:

As used herein, the term “scent infused” refers to the presence of a chemical compound in a structure, polymer, and/or formulation that is perceivable through olfaction upon emission, such as, e.g., diffusion. In some embodiments, emission of such chemical compound provides a pleasant scent.

As used herein, the term “emission period” refers to a period of time during which a chemical compound that is perceivable through olfaction (i.e., a scented compound) emits and/or diffuses from a structure. In some embodiments, the emission period is from about 1 to about 100 hours.

As used herein, the term “fluidic communication” refers to the flow and/or lack of flow of a fluid from a first position to a second position. When a fluid flows and/or is capable of flowing from a first position to a second position, the fluidic communication is open therebetween. When a fluid does not flow and/or is incapable of flowing from a first position to a second position, the fluidic communication is closed therebetween.

The terms “a,” “an,” and “the”, as used herein, refer to “one or more.” For example, reference to “a scent infused polymer” may include both reference to a single scent infused polymer and reference to a plurality of scent infused polymers.

Embodiments of the present disclosure relate to scent infused nasal cannulas, to scent infused nasal cannula assemblies, to methods of delivering scents to subjects during nasal delivery, and to methods of fabricating scent infused nasal cannulas. In some embodiments, the scent infused nasal cannulas described herein are used in a hospital setting (including both adult and child hospitals), such as, e.g., Emergency Room Departments, Burn Units, Cancer Units, Intensive Care Units, and/or Geriatric Care Units, etc. In some embodiments, the scent infused nasal cannulas described herein are used in a first response setting, such as, e.g., an ambulance.

In other embodiments, the scent infused nasal cannulas described herein are used outside of a hospital setting and/or a surgical setting only, and are not used in a hospital and/or a surgical setting. For example, the scent infused nasal cannulas described herein may be used in a nursing home setting and/or a residential setting without the need for medical supervision. In still other embodiments, the scent infused nasal cannulas described herein are used in recreational settings, such as, e.g., airline travelling and/or transportation settings.

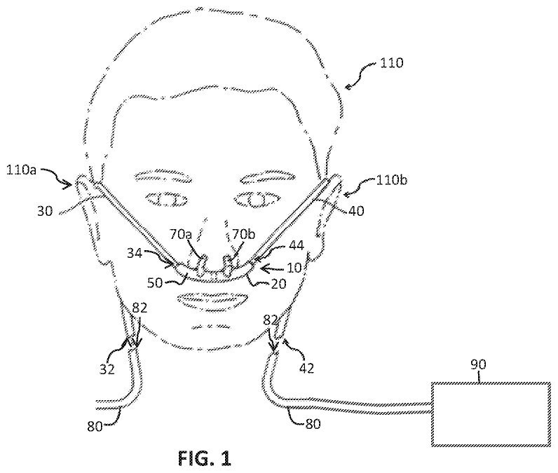

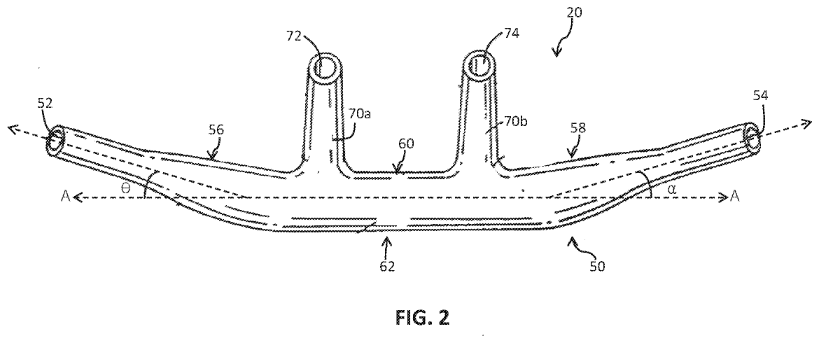

Embodiments of scent infused nasal cannulas will now be described in detail with reference to FIGS. 1 and 2. Thereafter, embodiments of scent infused nasal cannula assemblies will be described in detail.

### I. Scent Infused Nasal Cannulas

Referring to FIGS. 1 and 2, embodiments of a scent infused nasal cannula 10 for nasal delivery of a fluid from a fluid source to a subject 100 are depicted. The scent infused nasal cannula 10 may include a nose piece 20, a first fluid supply tube 30, and a second fluid supply tube 40.

Referring to FIG. 2, the nose piece 20 may include a tubular body 50 and a pair of narine projections 70a, 70b. With regard to the tubular body 50, the tubular body 50 may be hollow, defining a channel therein, and may define a first fluid inlet 52 and a second fluid inlet 54. In one or more embodiments, the tubular body 50 includes a first end section 56, a second end section 58, and a central section 60 extending between the first end section 56 and the second end section 58. In these embodiments, the first end section 56 defines the first fluid inlet 52 and the second end section 58 defines the second fluid inlet 54. In other embodiments, the first end section 56 or a portion thereof tapers from a surface in contact with the central section 60 of the tubular body 50 toward the first fluid inlet 52. Similarly, in other embodiments, the second end section 58 or a portion thereof tapers from a surface in contact with the central section 60 of the tubular body 50 toward the second fluid inlet 54. In some embodiments, the nose piece 20 is removable, such as e.g., without use of a tool, from the first fluid supply tube 30 and/or the second fluid supply tube 40 (described in greater detail below). In other embodiments, the nose piece 20 is removable from the first fluid supply tube 30 and/or the second fluid supply tube 40 such that it may be separated therefrom without modifying, significantly damaging, and/or destroying the structure of the nose piece 20, the first fluid supply tube 30 and/or the second fluid supply tube 40. In further embodiments, the nose piece 20 is disposable and/or single use, wherein the nose piece 20 may be replaced upon termination of the emission period and/or wherein a subject wearing such nose piece 20 may no longer perceive the scented compound.

In one or more embodiments, the central section 60 of the tubular body 50 is substantially straight. In further embodiments, the first end section 56 and the second end section 58 are bent relative to the central section 60 of the tubular body 50. For example, in these particular embodiments, the first end section 56 and the second end section 58 may extend at acute angles θ, α relative to a plane (A) bisecting the central section 60 of the tubular body 50 and being parallel to a bottom surface 62 of the central section 60 of the tubular body 50. More particularly, the angles θ, α may be respectively formed in between a plane (A) bisecting the central section 60 of the tubular body 50 and being parallel to the bottom surface 62 of the central section 60 of the tubular body 50 and axes of the first end section 56 of the tubular body 50 and the second end section 58 of the tubular body 50. Stated another way, the angle θ may be formed by intersection of a plane (A) bisecting the central section 60 of the tubular body 50 and being parallel to the bottom surface 62 of the central section 60 of the tubular body 50 and an axis of the first end section 56 of the tubular body 50. Similarly, the angle α may be formed by intersection of the plane (A) bisecting the central section 60 of the tubular body 50 and being parallel to the bottom surface 62 of the central section 60 of the tubular body 50 and an axis of the second end section 58 of the tubular body 50. In embodiments, the angles θ, α are independently from about 5° to about 60°, or from about 10° to about 55°, or from about 15° to about 50°, or from about 20° to about 45°, or from about 25° to about 40°, or about 30°.

Each of the first end section 56, the second end section 58, and the central section 60 may independently have an ovular, circular, or elliptical cross-sectional shape. In further embodiments, the tubular body 50 is configured to contact an upper lip region of a subject 100 wearing the scent infused nasal cannula 10.

The tubular body 50 may be made via methods known to one of ordinary skill in the art. The tubular body 50 may include and/or be formed from a scent infused polymer and/or formulation thereof known to those of ordinary skill in the art. For example, the tubular body 50 may include and/or be formed from a scent infused flexible polymer and/or a formulation thereof, such that tubular body 50 is flexible, soft and/or deformable. In embodiments, the scent infused flexible polymer is a scent infused medical grade polymer, such as e.g., a scent infused medical grade plastic polymer. In embodiments, the scent infused medical grade plastic is a scent infused thermoplastic polymer. In embodiments, the tubular body 50 is formed from a scent infused polysaccharide such as, e.g., a scent infused cellulosic polymer. In embodiments, the polymer into which a scented compound is infused and/or encapsulated is operable to release and/or facilitate release of the scented compound therefrom. In some embodiments, the polymer into which a scented compound is infused and/or encapsulated is substantially free of bisphenol A (i.e., BPA) and/or phthalates. Examples of suitable polymers into which a scented compound may be infused and/or encapsulated include, but are not limited to, thermoplastic polymers. In embodiments, the thermoplastic polymers are medical grade thermoplastic polymers. Examples of thermoplastic polymers suitable for use herein include acrylic polymers, such as, e.g., poly(methyl methacrylate), acrylonitrile butadiene styrene (i.e., ABS) polymers, nylon polymers, polylactic acid polymers, polybenzimidazole (i.e., PBI) polymers, polycarbonate polymers, polyether sulfone (i.e., PES) polymers, polyetherether ketone (i.e., PEEK) polymers, polyetherimide (i.e., PEI) polymers, polyethylene (i.e., PE) polymers, polyphenylene oxide (i.e., PPO) polymers, polyphenylene sulfide (i.e., PPS) polymers, polypropylene (i.e., PP) polymers, polystyrene polymers, polyvinyl chloride (i.e., PVC) polymers, teflon polymers, cellulosic polymers, thermoplastic elastomers (i.e., TPE), silicones, and combinations thereof. Examples of suitable TPE include styrenic block copolymers (i.e., TPE-s), polyolefin blends (i.e., TPE-o), elastomeric alloys (i.e., TPE-v or TPV), thermoplastic polyurethanes (i.e., TPU), thermoplastic copolyesters, thermoplastic polyamides, and combinations thereof.

A scented compound may be infused and/or encapsulated into a thermoplastic polymer. In embodiments, the scented compound is infused and/or encapsulated into a cellulosic polymer. In particular embodiments, the scented compound is infused and/or encapsulated into a cellulose ester. Examples of such cellulose esters include cellulose acetate butyrate, cellulose acetate propionate, cellulose acetate, and combinations thereof. In embodiments, the cellulosic polymer into which the scented compound is infused and/or encapsulated is a cellulose acetate (i.e., CA). In embodiments, the cellulosic polymer into which the scented compound is infused and/or encapsulated is cellulose acetate propionate (i.e., CAP). In embodiments, the cellulosic polymer into which the scented compound is infused and/or encapsulated is cellulose acetate butyrate (i.e., CAB).

In embodiments, cellulose acetate includes cellulose with acetyl groups grafted and/or attached thereon. In some embodiments, the cellulose includes from about 30% to about 45%, or from about 35% to about 40% by weight of acetyl groups, and from about 1% to about 10%, or from about 2% to about 9%, or from about 3% to about 8%, or from about 4% to about 7%, or from about 5% to about 6% by weight of hydroxyl groups per cellulose molecule. In one or more embodiments, the cellulose acetate has a weight average molecular weight of from than about 30,000 to about 50,000, as determined by gel permeation chromatography (hereinafter, “GPC”).

In embodiments, cellulose acetate propionate includes cellulose with acetyl groups and propionyl groups grafted and/or attached thereon. In some embodiments, the cellulose includes from about 0.5% to about 5.0%, or from about 1.0% to about 4.0%, or from about 2.0% to about 3.0% by weight of acetyl groups, from about 1% to about 10%, or from about 2% to about 9%, or from about 3% to about 8%, or from about 4% to about 7%, or from about 5% to about 6% by weight of hydroxyl groups, and from about 35% to about 55%, or from about 40% to about 50% by weight of propionyl groups per cellulose molecule. In one or more embodiments, the cellulose acetate propionate has a weight average molecular weight of from about 15,000 to about 75,000 as determined by GPC.

In embodiments, cellulose acetate butyrate includes cellulose with acetyl groups and butyryl groups grafted and/or attached thereon. In some embodiments, the cellulose includes from about 1% to about 40%, or from about 5% to about 35%, or from about 10% to about 30% or from about 15% to about 25% by weight of acetyl groups, from about 0.5% to about 5.0%, or from about 1.0% to about 4.0%, or from about 2.0% to about 3.0% by weight of hydroxyl groups, and from about 15% to about 55%, or from about 20% to about 50%, or from about 25% to about 45%, or from about 30% to about 40% by weight of butyryl groups per cellulose molecule. In one or more embodiments, the cellulose acetate butyrate has a weight average molecular weight of from about 12,000 to about 70,000 as determined by GPC.

In some embodiments, the polymer into which a scented compound may be infused and/or encapsulated is breathable, i.e., operable to allow passage of water vapor therethrough. In this way, upon infusion of a scented compound therein, such breathable scent infused cellulosic polymers may be operable to release and/or facilitate release of the scented compounds therefrom upon interaction with air and/or moisture, such as e.g., water vapor. In particular embodiments, cellulosic polymers into which a scented compound may be infused and/or encapsulated are breathable. In some embodiments, cellulosic polymers, such as, e.g., cellulose acetate, cellulose acetate propionate, and/or cellulose acetate butyrate, are warm to the touch.

In embodiments, a scented compound is infused into, encapsulated within, and/or otherwise combined with the polymers previously described via methods known to those of ordinary skill in the art. By way of example, as described in U.S. Pub. No. 2004/0028779, the contents of which are hereby incorporated by reference, scents have been added to polymers and/or plastics via the addition of polymer flavor concentrates (Naarden International, New York, N.Y.). The scented compound is infused into, encapsulated within, and/or added to the polymers in an amount sufficient to achieve a desired scent intensity, ranging from, e.g., subtle to intermediate to intense. The scented compounds infused into such polymers may provide a scent that is perceived to be pleasant. Suitable scented compounds are known to those of ordinary skill in the art.

In some embodiments, the scented compounds may be of the aldehydic, ambery, citrus, floral, fruity, gourmand, green, herbal, leather, marine, musky, spicy, and/or woody olfactive families, such as are produced by Givaudan (Cincinnati, Ohio). For example, in some embodiments, the scented compound provides a scent selected from the group consisting of fruit, mint, chocolate, nut, flower, and combinations thereof. Suitable fruit scents may include, but should not be limited to, blackberry, blueberry, strawberry, raspberry, apple, cherry, cranberry, orange, lemon, lime, grapefruit, pear, pumpkin, melon, coconut, kiwi, pineapple, mango, banana, passion fruit, and combinations thereof. Suitable mint scents may include, but should not be limited to, spearmint, peppermint, and combinations thereof. Suitable chocolate scents may include, but should not be limited to, milk chocolate, dark chocolate, chocolate fudge, white chocolate, and combinations thereof. Suitable nut scents may include, but should not be limited to, almond, cashew, hazlenut, pecan, peanut, and combinations thereof. Suitable flower scents may include, but should not be limited to, chamomile, roses, lavender, gardenia, hydrangea, plumeria, and combinations thereof. By way of specific example, the following represent suitable scents and associated scented compounds:

TABLE I
Scented Compounds

| OLFACTIVE | | |
|---|---|---|
| NOTE/SCENT | SCENTED COMPOUND | MANUFACTURER |
| Strawberry | Ethyl 3-phenyl- | Givaudan |
| | 2,3-epoxybutanoate | (Cincinnati, OH) |
| | (Strawberry Pure) | |
| Raspberry | Ethyl 6-acetoxyhexanoate | Givaudan |
| | (Berryflor ®) | (Cincinnati, OH) |
| Peach | 2-Methyldecanenitrile | Givaudan |
| | (Fructonile) | (Cincinnati, OH) |
| Apple/Pineapple | Ethyl 2-methylpentanoate | Givaudan |
| | (Manzanate) | (Cincinnati, OH) |
| Pear/Pineapple | Isopropyl Methyl-2-Butyrate | Givaudan |
| | (Isopropyl Methyl-2-Butyrate) | (Cincinnati, OH) |
| Tropical Fruit | 2-Ethyl-N-methyl-N-(3- | Givaudan |
| | methylphenyl)butanamide | (Cincinnati, OH) |
| | (Paradisamide ®) | |

In some embodiments, the scent infused polymer is provided in a scent infused polymer formulation. In one or more particular embodiments, the scent infused polymer formulation is substantially free of bisphenol A (i.e., BPA) and/or phthalates. In embodiments, the scent infused polymer formulation includes a scent infused polymer, a plasticizer, a stabilizer, and/or a colorant.

In some embodiments, the scent infused polymer formulation includes a polymer as previously described. In particular embodiments, the polymer is a cellulosic polymer, such as, e.g., cellulose acetate, cellulose acetate propionate, cellulose acetate butyrate, or combination thereof. In embodiments, the scent infused polymer formulation includes a polymer in an amount sufficient to form the tubular body 50 and/or to encapsulate a scented compound. In some embodiments, the scent infused polymer formulation includes from about 30% to about 90%, or from about 40% to about 80%, or from about 45% to about 75%, or greater than about 50% by weight of a polymer into which a scented compound may be infused. In further embodiments, the scent infused polymer formulation includes greater than about 75% by weight of a polymer into which a scented compound may be infused.

In some embodiments, the scent infused polymer formulation includes a plasticizer. Suitable plasticizers are known to those of ordinary skill in the art. In embodiments, the plasticizer is a phthalate, an adipate, a sebacate, a maleate, an acetate, or combination thereof. Specific examples of plasticizers include benzyl butyl phthalate, dicyclohexyl phthalate, diheptyl phthalate, diisobutyl phthalate, dimethyl phthalate, dioctyl terephthalate, diphenyl phthalate, dipropyl phthalate, diethyl phthalate, bis[2-(2-butoxyethoxy)ethyl] adipate, bis(2-ethylhexyl) adipate, dibutyl adipate, diethyl adipate, diisobutyl adipate, diisodecyl adipate, dimethyl adipate, dioctyl adipate, triacetin, diacetin, and combinations thereof. In one or more embodiments, the plasticizer is provided in an amount sufficient to form the tubular body 50 and/or to appropriately modulate the plasticity thereof. In specific embodiments, wherein the polymer is a cellulosic polymer, such as, e.g., cellulose acetate, cellulose acetate propionate, and/or cellulose acetate butyrate, the plasticizer is chosen from diethyl phthalate, triacetin, dioctyl adipate, diacetin, or combination thereof. In some embodiments, the scent infused polymer formulation includes from about 0% to about 50%, or from about 5% to about 45%, or from about 10% to about 40%, or from about 15% to about 35%, or from about 20% to about 30% by weight of a plasticizer.

In further embodiments, the scent infused polymer formulation includes a stabilizer and/or a solubilizing compound. Suitable stabilizers and/or solubilizing compounds are known to those of ordinary skill in the art. In embodiments, the stabilizing and/or solubilizing compound is hexylene glycol. In one or more embodiments, the stabilizer and/or solubilizing compound is provided in an amount sufficient to form the tubular body 50 and/or to stabilize and/or solubilize the formulation and/or components thereof. In embodiments, the scent infused polymer formulation includes from about 10% to about 30%, or from about 15% to about 25% by weight of a stabilizer. In embodiments, the scent infused polymer formulation includes less than about 15% by weight of a stabilizer. In other embodiments, the scent infused polymer formulation includes less than about 10% by weight of a stabilizer.

In one or more embodiments, the scent infused polymer formulation includes a scented compound, such as was previously described. The scent infused polymer formulation includes a scented compound in an amount sufficient to achieve a desired scent intensity. In some embodiments, a scent infused polymer formulation is loaded with a scented compound in an amount sufficient to achieve a subtle scent intensity. In other embodiments, a scent infused polymer formulation is loaded with a scented compound in an amount sufficient to achieve an intense scent intensity. In embodiments, the scent infused polymer formulation includes less than about 40% by weight of the scented compound, or from about 0.1% to about 39.9% by weight of the scented compound. In some embodiments, the scent infused polymer formulation includes less than about 30% by weight of the scented compound, or from about 0.1% to about 29.9% by weight of the scented compound. In other embodiments, the scent infused polymer formulation includes less than about 20% by weight of the scented compound, or from about 0.1% to about 19.9% by weight of the scented compound. In still other embodiments, the scent infused polymer formulation includes less than about 10% by weight of the scented compound, or from about 0.1% to about 9.9% by weight of the scented compound.

In one or more embodiments, the scent infused polymer formulation includes a colorant. Suitable colorants are known to those of ordinary skill in the art. The scent infused polymer formulation includes a colorant in an amount sufficient to achieve a desired color. In some embodiments, the scent infused polymer formulation includes less than about 10% by weight of the colorant, or from about 0.1% to about 9.9% by weight of the colorant. In still further embodiments, the scent infused polymer formulation includes less than about 5% by weight of the colorant, or from about 0.1% to about 4.9% by weight of the colorant.

A suitable commercial embodiment of a scent infused polymer formulation includes, but is not limited to, Auracell® CA, Auracell® CAB, and/or Auracell® CAP produced by Rotuba Extruders, Inc. (Linden, N.J.). Product component information for each of such commercial embodiments is set forth in Tables II-IV below:

TABLE II
Fragranced Cellulose Acetate Plastic Pellets Auracell ® CA

| COMPONENTS | APPROXIMATE WEIGHT % |
|---|---|
| Cellulose Acetate | >50 |
| Diethyl Phthalate* | 0-40 |
| Triacetin* | 0-40 |
| Diacetin* | 0-40 |
| Hexylene Glycol | <15 |
| Scented Compound | <30 |
| Colorants/Additives | <10 |

*Specific formulations may be specified with either diethyl phthalate or triacetin, or a mixture of these plasticizers.

TABLE III
Fragranced Cellulose Acetate Butyrate Plastic
Pellets Auracell ® CAB

| COMPONENT | APPROXIMATE WEIGHT % |
|---|---|
| Cellulose Acetate Butyrate | >75 |
| Diethyl Phthalate* | 0-20 |
| Dioctyl Adipate* | 0-20 |
| Hexylene Glycol | <10 |
| Scented Compound | <20 |
| Colorants/Additives | <10 |

*Specific formulations may be specified with either diethyl phthalate or dioctyl adipate, or a mixture of these plasticizers.

TABLE IV
Fragranced Cellulose Acetate Propionate Plastic
Pellets Auracell ® CAP

| COMPONENTS | APPROXIMATE WEIGHT % |
|---|---|
| Cellulose Acetate Propionate | >75 |
| Diethyl Phthalate* | 0-20 |
| Dioctyl Adipate* | 0-20 |
| Hexylene Glycol | <10 |
| Scented Compound | <20 |
| Colorants/Additives | <10 |

*Specific formulations may be specified with either diethyl phthalate or dioctyl adipate, or a mixture of these plasticizers.

Referring still to FIG. 2, the pair of narine projections 70a, 70b of the tubular body 50 may define a first fluid outlet 72 and a second fluid outlet 74. In one or more embodiments, the narine projections 70a, 70b are hollow, defining channels therein, integral with the tubular body 50, extending outwardly from, and/or substantially normal to the tubular body 50. In still other embodiments, the narine projections 70a, 70b independently have an ovular, circular, or elliptical cross-sectional shape. In further embodiments, the narine projections 70a, 70b are configured for insertion within the nares of the subject 100 wearing the scent infused nasal cannula 10. For example, in some embodiments, the narine projections 70a, 70b are spaced apart such that they are insertable within nares of the subject 100 wearing the scent infused nasal cannula 10. In other embodiments, the narine projections 70a, 70b are spaced apart such that at least a portion thereof is insertable within the nares of the subject 100 wearing the scent infused nasal cannula 10. In one or more embodiments, the narine projections 70a, 70b are formed from a scent infused polymer and/or formulation thereof as previously described with regard to the tubular body 50.

Referring to FIG. 1 again, the first fluid supply tube 30 and the second fluid supply tube 40 may be respectively attachable to the first fluid inlet 52 and the second fluid inlet 54 of the tubular body 50. In some embodiments, the first fluid supply tube 30 includes a first supply inlet 32 and a first supply outlet 34. Similarly, in other embodiments, the second fluid supply tube 40 includes a second supply inlet 42 and a second supply outlet 44. In one or more embodiments, the first fluid supply tube 30 and the second fluid supply tube 40 are also each attachable to the fluid source. In this way, the first fluid supply tube 30 and the second fluid supply tube 40 may be in fluidic communication, such as, e.g., open fluidic communication, with the tubular body 50 (and with the narine projections 70a, 70b) for delivery of the fluid from the fluid source to the subject 100 wearing the scent infused nasal cannula 10.

In some embodiments, the first fluid supply tube 30 and the second fluid supply tube 40 are independently formed from a flexible polymer, such that the first supply tube 30 and the second supply tube 40 are flexible, soft, and/or deformable. In embodiments, the flexible polymer is a medical grade polymer, such as e.g., a medical grade plastic. Examples of suitable polymers include, but are not limited to, thermoplastic polymers. In embodiments, the thermoplastic polymers are medical grade thermoplastic polymers. Examples of thermoplastic polymers suitable for use herein include acrylic polymers, such as, e.g., poly(methyl methacrylate), acrylonitrile butadiene styrene (i.e., ABS) polymers, nylon polymers, polylactic acid polymers, polybenzimidazole (i.e., PBI) polymers, polycarbonate polymers, polyether sulfone (i.e., PES) polymers, polyetherether ketone (i.e., PEEK) polymers, polyetherimide (i.e., PEI) polymers, polyethylene (i.e., PE) polymers, polyphenylene oxide (i.e., PPO) polymers, polyphenylene sulfide (i.e., PPS) polymers, polypropylene (i.e., PP) polymers, polystyrene polymers, polyvinyl chloride (i.e., PVC) polymers, teflon polymers, cellulosic polymers, thermoplastic elastomers (i.e., TPE), silicones, and combinations thereof. In further embodiments, the fluid supply tubes 30, 40 may be formed from a scent infused polymer and/or formulation thereof, as previously described with regard to the tubular body 50.

The scent infused nasal cannula 10 may be formed via methods known to one of ordinary skill in the art. More particularly, in some embodiments, the scent infused nasal cannula 10 is formed via methods known to one of ordinary skill in the art from scent infused polymers and/or formulations thereof, such as were previously described. In some embodiments, the scent infused nasal cannula 10 is formed via injection molding, dip molding, and/or additional molding techniques known to one of ordinary skill in the art. In some embodiments, the scent infused nasal cannula 10 is formed via an injection molding process from a scent infused polymer and/or formulation thereof, such as is known to one of ordinary skill in the art. In further embodiments, the scent infused nasal cannula 10 is formed via an injection molding process from a cellulosic polymer, such as, e.g., a cellulosic acetate polymer formulation Auracell® CA which is commercially available from Rotuba.

Where a cellulosic polymer formulation, such as, e.g., Cellulose Acetate Plastic Pellets Auracell® CA, Cellulose Acetate Plastic Pellets Auracell® CAB, or Cellulose Acetate Propionate Plastic Pellets Auracell® CAP, is employed, suitable molding conditions and pressures associated therewith are as set forth in Tables V-VII below:

TABLE V
Cellulose Acetate Starting Barrel Temperature

| FLOW | MSS (° F.) | MS (° F.) | M (° F.) | MH (° F.) | H (° F.) | H2 (° F.) | H3 (° F.) |
|---|---|---|---|---|---|---|---|
| Rear | 350-370 | 340-360 | 360-380 | 380-400 | 400-420 | 420-450 | 450-470 |
| Center | 350-370 | 340-360 | 360-380 | 380-400 | 400-420 | 420-450 | 450-470 |
| Front | 350-370 | 340-360 | 360-380 | 380-400 | 400-420 | 420-450 | 450-470 |
| Nozzle | 370-390 | 360-380 | 380-400 | 390-410 | 410-430 | 430-460 | 460-480 |

TABLE VI
Cellulose Acetate Propionate Barrel Temperature Starting Profile

| FLOW | MH (° F.) | H (° F.) | H2 (° F.) | H3 (° F.) |
|---|---|---|---|---|
| Root | 280-290 | 290-300 | 300-310 | 315-325 |
| Rear - Root | 290-300 | 300-310 | 310-320 | 325-335 |
| Rear - Center | 300-310 | 310-320 | 320-330 | 335-345 |
| Center | 325-335 | 335-345 | 345-355 | 360-370 |
| Front | 355-375 | 365-385 | 375-395 | 390-410 |
| Nozzle | 355-375 | 365-385 | 375-395 | 390-410 |

TABLE VII
Pressures

| FLOW | MSS | MS | M | MH | H | H2 | H3 |
|---|---|---|---|---|---|---|---|
| Moveable | 1100/ | 1200/ | 1300/ | 1400/ | 1500/ | 1600/ | 1700/ |
| Half | 950 | 1000 | 1100 | 1100 | 1200 | 1200 | 1300 |
| | PSIG | PSIG | PSIG | PSIG | PSIG | PSIG | PSIG |
| Back | 0-50 | 0-50 | 0-50 | 0-50 | 0-50 | 0-50 | 0-50 |
| Pressure | PSI | PSI | PSI | PSI | PSI | PSI | PSI |

Moreover, with regard to mold temperature, in embodiments, soft flows are below MS −100° F.-140° F. Harder flows may be from about −100° F.-180° F. With regard to cycle times:
INJECTION—8-12 sec.
BOOSTER—6-12 sec.
CURE—40-70 sec.

With regard to drying conditions, in embodiments, desiccant dryers may be used at reduced temperatures and drying times. For example, 150° F. for 2 hours is a target with a dew point of between about −20° F. and about −40° F. It is noted that, in some embodiments, too high of temperatures and drying times can burn out the fragrance (i.e., scent) of the scented compound. In the absence of a desiccant dryer, an injection molding machine hopper may be used. Tray dryers may also be used.

With regard to tooling, in embodiments, the mold vent for the tool is from about 0.002″ to about 0.003″. If this is not the case, mold temperatures may be elevated to help drive off volatiles thereby reducing surface blushing. (180° F.-220° F.)

With regard to mold temperature regulation, in embodiments, most molding thermolators use hot water as a medium. In the case of elevated mold temperatures as previously described, oil thermolators may be used to achieve high temperatures of ≥220° F. without turning to steam.

Additional guidelines are as follows: (1) Use straight flow through or revers nozzle. Nozzle orifice may be 1/16″ smaller than sprue orifice at end of mold; (2) Do not exceed 160° F. without oil medium. Higher mold temps may be used to burn off surface plasticizer. In embodiments, a thermolator is used to heat mold or clean water (just cracked). In embodiments, a chiller is not be used; and (3) Injection time may be increased; depending upon part thickness, part size, and number of cavities.

In some embodiments, the subject is a mammal. In some particular embodiments, the subject is a human, including, e.g., adults and children. In other embodiments, the fluid is a gas. In still other embodiments, the fluid is oxygen. Additionally, the fluid source 90 may be an oxygen supply source. In some embodiments, the fluid source 90 is an oxygen reservoir. In other embodiments, the fluid source 90 is an oxygen generator.

In some embodiments, the nose piece 20, the first fluid supply tube 30, and/or the second fluid supply tube 40 are at least one of transparent or opaque. In other embodiments, the nose piece 20, the first fluid supply tube 30, and/or the second fluid supply tube 40 are colored. Examples of suitable colors include red, orange, yellow, green, blue, indigo, violet, brown, and combinations thereof. In further embodiments, the nose piece 20, the first fluid supply tube 30, and/or the second fluid supply tube 40 may be a color which corresponds to the scent emitted from the olfactory chemical compound. For example, suitable scents with corresponding colors may be as provided in Table VIII below:

TABLE VIII
Scents and Corresponding Colors

| SCENT | CORRESPONDING COLOR |
|---|---|
| Blackberry | Indigo |
| Blueberry | Blue |
| Strawberry | Red |
| Raspberry | Red |
| Apple | Green |
| Cherry | Red |
| Cranberry | Red |
| Orange | Orange |
| Lemon, | Yellow |
| Lime | Green |
| Grapefruit | Orange |
| Pear | Green |
| Pumpkin | Orange |
| Melon | Orange |
| Coconut | White |
| Kiwi | Green |
| Pineapple | Yellow |
| Mango | Orange |
| Banana | Yellow |
| Passion Fruit | Violet |
| Spearmint | Green |
| Peppermint | Green |
| Almond | Brown |
| Cashew | Brown |
| Hazlenut | Brown |
| Pecan | Brown |
| Peanut | Brown |
| Chamomile | White |
| Roses | Red |
| Lavender | Purple |
| Gardenia | White |
| Hydrangea | Blue |
| Plumeria | White |

Embodiments of scent infused nasal cannulas 10 have been described in detail. Embodiments of scent infused nasal cannula assemblies will now be described in detail.

### II. Scent Infused Nasal Cannula Assemblies

A scent infused nasal cannula assembly for nasal delivery of a fluid from a fluid source to a subject 100 may include a nose piece 20, a first fluid supply tube 30, a second fluid supply tube 40, and a third fluid supply tube 80. In some embodiments, the fluid, the fluid source, the subject 100, the nose piece 20, first fluid supply tube 30, and second fluid supply tube 40 are as previously described with regard to scent infused nasal cannulas 10.

The third fluid supply tube 80 may include a third supply outlet 82. In one or more embodiments, the third supply outlet 82 may be attached to the first supply inlet 32 and the second supply inlet 42. In this way, the first fluid supply tube 30, the second fluid supply tube 40, and the third fluid supply tube 80 may be in fluidic communication with the nose piece 20 for delivery of the fluid from the fluid source 90 to the subject 100 wearing the scent infused nasal cannula 10. The third fluid supply tube 80 may be formed from a flexible polymer, such as was previously described with regard to the first fluid supply tube 30 and the second fluid supply tube 40. In further embodiments, the third fluid supply tube 80 may be formed from a scent infused polymer and/or formulation thereof as was previously described with regard to the tubular body 50.

In some embodiments, the first fluid supply tube 30 and the second fluid supply tube 40 are attached to the third fluid supply tube 80 with a connector (not shown). In other embodiments, the assembly may also include a slip loop (not shown) disposed about the first fluid supply tube 30 and the second fluid supply tube 40.

Embodiments of scent infused nasal cannula assemblies have been described in detail. Embodiments of methods for providing a scent to a subject 100 during nasal delivery of a fluid from a fluid source to the subject 100 and for utilizing a scent infused nasal cannula 10 will now be described with reference to FIG. 1.

### III. Methods for Providing Scent to Subjects and Utilizing a Scent Infused Nasal Cannula

A method for providing a scent to a subject 100 during nasal delivery of a fluid from a fluid source may include attaching a scent infused nasal cannula 10 to the subject 100 and delivering the fluid from the fluid source to the subject 100. In some embodiments, the subject, the fluid, the fluid source, and the scent infused nasal cannula 10 are as previously described. Referring to FIG. 1, in some embodiments, the scent infused nasal cannula attached to the subject 100 includes a nose piece 20 and a pair of fluid supply tubes (e.g., first and second fluid supply tubes 30, 40) in open fluidic communication with the nose piece 20 and the fluid source 90.

In some embodiment, the scent infused nasal cannula 10 is attached to the subject 100 by releasably attaching the pair of supply tubes (i.e., the first and second fluid supply tubes 30, 40) to the subject's ears 110a, 110b. More specifically, the scent infused nasal cannula 10 is attached to the subject 100 by mounting the first and second supply tubes 30, 40 respectively over the subject's ears 110, 110b and then optionally guiding the first and second supply tubes 30, 40 along the sides of the subject's neck in front of the subject or extending to the back of the subject's head.

The fluid from the fluid source may be delivered to the subject 100 via the scent infused nasal cannula 10. The fluid may be delivered during at least a portion of the emission period of the scent infused polymer such that the scent is detectable therefrom. The scent may be detected qualitatively and/or quantitatively. In some embodiments, the fluid includes oxygen and/or aerosol therapeutics. In one or more embodiments wherein the fluid is oxygen, oxygen is delivered to the subject 100 at a rate of from about 1 to about 60 liters/minute. In other embodiments wherein the fluid is oxygen, the oxygen is delivered to the subject 100 at a rate of from about 1 to about 5 liters/minute.

In some embodiments, a scent is provided to a subject 100 during nasal delivery of a fluid from a fluid source through a scent infused nasal cannula 10 in an environment outside of a hospital and/or outside of a surgical setting.

In some embodiments, methods for providing a scent to a subject 100 during nasal delivery via a scent infused nasal cannula 10 function to improve everyday medication uses, such as e.g., supplemental oxygen and/or therapeutics. For example, by delivering a fluid such as, e.g., supplemental oxygen and/or therapeutics, to the subject 100 while providing a scent thereto, the scent infused nasal cannula 10 may be operable to reduce dosages of therapeutics delivered thereto, to reduce tolerance to certain therapeutics delivered thereto (e.g., this is especially relevant to habit forming therapeutics, e.g., Vicodin), to reduce stress levels of the subject to whom the fluid is delivered, to improve the mood of the subject to whom the fluid is delivered, to improve the immune system of the subject to whom the fluid is delivered, to enhance the appetite of the subject to whom the fluid is delivered, and/or to suppress the appetite of the subject to whom the fluid is delivered (e.g., relative to delivery of fluids to a subject via traditional, unscented, nasal cannulas).

Uses of the scent infused nasal cannula 10 to achieve such results are also contemplated herein. For example, methods for reducing dosages of therapeutics delivered via a scent infused nasal cannula 10, methods for reducing tolerance to therapeutics delivered via a scent infused nasal cannula 10, methods for reducing stress of a subject to whom a fluid is delivered via a scent infused nasal cannula 10, methods for improving the immune system of a subject to whom a fluid is delivered via a scent infused nasal cannula 10, methods for enhancing the appetite of a subject to whom a fluid is delivered via a scent infused nasal cannula 10, and/or methods for suppressing the appetite of a subject to whom a fluid is delivered via a scent infused nasal cannula 10 are also contemplated herein. Such methods also include attaching a scent infused nasal cannula 10 to the subject 100 and delivering the fluid from the fluid source to the subject 100.

Embodiments of methods for providing a scent to a subject 100 during nasal delivery of a fluid from a fluid source to the subject 100 and for utilizing a scent infused nasal cannula 10 have been described in detail. Embodiments of fabricating a scent infused nasal cannula for nasal delivery of a fluid from a fluid source to the subject 100 will now be described.

### IV. Methods of Fabricating Scent Infused Nasal Cannulas

A method of fabricating a scent infused nasal cannula 10 for nasal delivery of a fluid from a fluid source to a subject 100 may include providing a scent infused nose piece 20 and attaching a pair of fluid supply tubes (e.g., the first and second fluid supply tubes 30, 40) to the nose piece 20 and to the fluid source. The fluid, fluid source, subject 100, nose piece 20, and first and second fluid supply tubes 30, 40 are as previously described. In some particular embodiments, the scent infused nose piece 20 includes a tubular body 50 and a pair of narine projections 70a, 70b integral and in fluidic communication, such as, e.g., open fluidic communication, therewith.

The pair of fluid supply tubes (e.g., the first and second fluid supply tubes 30, 40) may be attached to the first fluid inlet 52 of the tubular body 50, the second fluid inlet 54 of the tubular body 50, and the fluid source 90.

Embodiments of fabricating a scent infused nasal cannula for nasal delivery of a fluid from a fluid source to the subject 100 have now been described. Embodiments of a scent infused nasal cannula kit will now be described.

### V. Scent Infused Nasal Cannula Kits

A scent infused nasal cannula kit may include at least one scent infused nasal cannula 10 as previously described. In some embodiments, the kit may include scent infused nasal cannulas 10 of varying sizes, scents, and/or colors. For example, an adult or large-sized scent infused nasal cannula 10 may be used for adults and a child or small-sized scent infused nasal cannula 10 may be used for children. In further embodiments, the kit may also include fluid supply tubes 30, 40.

In one or more embodiments, the kit may include a case for accommodating the scent infused nasal cannula 10 and/or the supply tubes 30, 40. Specifically, the case may include an upper housing attached to a lower housing for accommodating the scent infused nasal cannula 10 and/or the supply tubes 30, 40. In one particular embodiment, the upper housing is hingedly attached to the lower housing with a hinging mechanism. The case may include an open configuration and a closed configuration (not shown).

It should now be understood that various aspects of the scent infused nasal cannula, assemblies, and methods of use and fabrication thereof are described herein and that such aspects may be utilized in conjunction with various other aspects.

In a first aspect, the disclosure provides a scent infused nasal cannula for nasal delivery of nasal fluids from fluid sources to subjects are disclosed. Such cannula includes a tubular body defining a first fluid inlet and a second fluid inlet, a pair of narine projections defining a first fluid outlet and a second fluid outlet, a first fluid supply tube, and a second fluid supply tube. The pair of narine projections are in fluidic communication with the tubular body and are integral with and outwardly extending from the tubular body. The tubular body and the pair of narine projections are formed from a scent infused polymer from which a scent emits for an emission period. The first fluid supply tube is attachable to the first inlet of the tubular body and is attachable to the fluid source. The second fluid supply tube is attachable to the second inlet of the tubular body and is attachable to the fluid source. When the first fluid supply tube and the second fluid supply tube are attached to the first inlet of the tubular body, to the second inlet of the tubular body, and to the fluid source, the first fluid supply tube and the second fluid supply tube are in fluidic communication with the tubular body and with the fluid source for delivery of the fluid from the fluid source to the subject.

In a second aspect, the disclosure provides a cannula of the first aspect, in which the tubular body includes a first end section, a second end section, and a central section extending between the first end section and the second end section, and the first end section and the second end section respectively define the first fluid inlet and the second fluid inlet.

In a third aspect, the disclosure provides a cannula of the first or second aspect, in which the tubular body includes a first end section, a second end section, and a central section extending between the first end section and the second end section, the first end section and the second end section respectively define the first fluid inlet and the second fluid inlet, and the first end section and the second end section extend at respective angles θ, α relative to a horizontal plane A of the tubular body.

In a fourth aspect, the disclosure provides a cannula of any of the first to the third aspects, in which the tubular body includes a first end section, a second end section, and a central section extending between the first end section and the second end section, the first end section and the second end section respectively define the first fluid inlet and the second fluid inlet, and the first end section and the second end section are bent relative to the central section of the tubular body.

In a fifth aspect, the disclosure provides a cannula of any of the first to the fourth aspects, in which the tubular body includes a first end section, a second end section, and a central section extending between the first end section and the second end section, and the central section is substantially straight.

In a sixth aspect, the disclosure provides a cannula of any of the first to the fifth aspects, in which tubular body includes a first end section, a second end section, and a central section extending between the first end section and the second end section, the first end section and the second end section respectively define the first fluid inlet and the second fluid inlet, the first end section tapers from a surface thereof in contact with the central section to the first fluid inlet, and the second end section tapers from a surface thereof in contact with the central section to the second fluid inlet.

In a seventh aspect, the disclosure provides a cannula of any of the first to the sixth aspects, in which the tubular body is configured to contact an upper lip region of the subject.

In an eighth aspect, the disclosure provides a cannula of any of the first to the seventh aspects, in which the pair of narine projections are configured for insertion within nares of the subject for nasal delivery of the fluid.

In a ninth aspect, the disclosure provides a cannula of any of the first to the eighth aspects, in which the pair of narine projections are spaced apart such that the narine projections are insertable within nares of the subject for the nasal delivery of the fluid.

In a tenth aspect, the disclosure provides a cannula of any of the first to the ninth aspects, in which the pair of narine projections are spaced apart such that at least a portion of the narine projections are insertable into nares of the subject for the nasal delivery of the fluid.

In an eleventh aspect, the disclosure provides a cannula of any of the first to the tenth aspects, in which the scent infused polymer is selected from the group consisting of a polyethylene polymer, a polyethylene copolymer, a thermoplastic rubber, and a silicone.

In a twelfth aspect, the disclosure provides a cannula of any of the first to the eleventh aspects, in which the scent infused polymer is flexible.

In a thirteenth aspect, the disclosure provides a cannula of any of the first to the twelfth aspects, in which the scent which emits from the scent infused polymer is selected from the group consisting of fruit, mint, chocolate, nut, flower, and combinations thereof.

In a fourteenth aspect, the disclosure provides a cannula of any of the first to the thirteenth aspects, in which the tubular body and the pair of narine projections are at least one of transparent or opaque.

In a fifteenth aspect, the disclosure provides a cannula of any of the first to the fourteenth aspects, in which the tubular body and the pair of narine projections are at least one of red, orange, yellow, green, blue, indigo, violet, or brown.

In a sixteenth aspect, the disclosure provides a cannula of any of the first to the fifteenth aspects, in which the tubular body and the pair of narine projections are a color which corresponds to the scent.

In a seventeenth aspect, the disclosure provides a cannula of any of the first to the sixteenth aspects, in which the emission period is from about 1 hour to about 100 hours.

In an eighteenth aspect, the disclosure provides a cannula of any of the first to the seventeenth aspects, in which the fluid source is an oxygen supply source.

In a nineteenth aspect, the disclosure provides a cannula of any of the first to the eighteenth aspects, in which the fluid source is an oxygen reservoir.

In a twentieth aspect, the disclosure provides a cannula of any of the first to the nineteenth aspects, in which the fluid source is an oxygen generator.

In a twenty-first aspect, the disclosure provides a scent infused nasal cannula assembly for nasal delivery of a fluid from a fluid source to a subject, the cannula assembly including a tubular body defining a first fluid inlet and a second fluid inlet, a pair of narine projections defining a first fluid outlet and a second fluid outlet, a first fluid supply tube including a first supply inlet and a first supply outlet, a second fluid supply tube including a second supply inlet and a second supply outlet, and a third fluid supply tube attached to the supply source and including a third supply outlet. The pair of narine projections are in fluidic communication with the tubular body and are integral with and outwardly extending from the tubular body. The tubular body and the pair of narine projections are formed from a scent infused polymer from which a scent emits for an emission period. The first supply outlet is attached to the first fluid inlet of the tubular body, the second supply outlet is attached to the second fluid inlet of the tubular body, and the third supply outlet is attached to the first supply inlet and the second supply inlet.

In a twenty-second aspect, the disclosure provides a cannula assembly of the twentieth aspect, in which the first fluid supply tube and the second fluid supply tube are attached to the third fluid supply tube with a connector.

In a twenty-third aspect, the disclosure provides a cannula assembly of the twentieth or the twenty-first aspects, in which a slip loop is disposed about the first fluid supply tube and the second fluid supply tube.

In a twenty-fourth aspect, the disclosure provides a method for providing a scent to a subject during nasal delivery of fluids from fluid sources to the subject. The method includes attaching a scent infused nasal cannula to the subject and delivering the fluid from the fluid source to the subject via the scent infused nasal cannula. The scent infused nasal cannula includes a nose piece and a pair of fluid supply tubes in fluidic communication with the nose piece and in fluidic communication with the fluid source. The nose piece is formed from a scent infused polymer from which a scent emits for an emission period. The fluid is delivered during at least a portion of the emission period of the scent infused polymer such that the scent is detectable therefrom.

In a twenty-fifth aspect, the disclosure provides a method of the twenty-fourth aspect, in which the fluid is a gas.

In a twenty-sixth aspect, the disclosure provides a method of the twenty-fourth to the twenty-fifth aspects, in which the fluid is oxygen.

In a twenty-seventh aspect, the disclosure provides a method of any of the twenty-fourth to the twenty-sixth aspects, in which the fluid is oxygen, and the oxygen is delivered to the subject at a rate of from about 1 to about 60 liters/minute.

In a twenty-eighth aspect, the disclosure provides a method of any of the twenty-fourth to the twenty-seventh aspects, in which the fluid is oxygen, and the oxygen is delivered to the subject at a rate of from about 1 to about 5 liters/minute.

In a twenty-ninth aspect, the disclosure provides a method of any of the twenty-fourth to the twenty-eighth aspects, in which the scent infused nasal cannula is attached to the subject by releasably attaching the pair of supply tubes to the subject's ears.

In a thirtieth aspect, the disclosure provides a method of fabricating a scent infused nasal cannula for nasal delivery of a fluid from a fluid source to a subject. The method includes providing a scent infused nose piece including a tubular body and a pair of narine projections integral with and in fluidic communication with the tubular body and attaching a pair of fluid supply tubes to the first inlet of the tubular body, to the second inlet of the tubular body, and to the fluid source. The tubular body defines a first fluid inlet and a second fluid inlet. The pair of narine projections define a first fluid outlet and a second fluid outlet. The scent infused nose piece is formed from a scent infused polymer from which a scent emits for an emission period.

It is noted that terms like “preferably,” “generally,” “commonly,” and “typically” are not utilized herein to limit the scope of the claims or to imply that certain features are critical, essential, or even important to the structure or function of the claims. Rather, these terms are merely intended to highlight alternative or additional features that may or may not be utilized in a particular embodiment of the present disclosure.

For the purposes of describing and defining the present disclosure it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

All documents cited are incorporated herein by reference; the citation of any document is not to be construed as an admission that it is prior art with respect to the present disclosure.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present disclosure have been illustrated and described, it would be obvious to one skilled in the art that various other changes and modifications can be made without departing from the spirit and scope of the disclosure. It is therefore intended to cover in the appended claims all such changes and modifications that are within the scope of this disclosure.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

What is claimed is:

1. A scent infused nasal cannula for nasal delivery of a fluid from a fluid source to a subject, the cannula comprising:
a tubular body defining a first fluid inlet and a second fluid inlet;
a pair of narine projections defining a first fluid outlet and a second fluid outlet, wherein the pair of narine projections are in fluidic communication with the tubular body, wherein the pair of narine projections are integral with and outwardly extending from the tubular body, and wherein the tubular body and the pair of narine projections are formed from a scent infused polymer from which a scent emits for an emission period;
a first fluid supply tube attachable to the first fluid inlet of the tubular body and attachable to the fluid source; and
a second fluid supply tube attachable to the second fluid inlet of the tubular body and attachable to the fluid source, such that:
when respectively attached to the first inlet of the tubular body, the second inlet of the tubular body, and the fluid source, the first fluid supply tube and the second fluid supply tube are in fluidic communication with the tubular body and with the fluid source for delivery of the fluid from the fluid source to the subject.

2. The cannula of claim 1, wherein:
the tubular body comprises a first end section, a second end section, and a central section extending between the first end section and the second end section; and
the first end section and the second end section respectively define the first fluid inlet and the second fluid inlet.

3. The cannula of claim 1, wherein the scent infused polymer is a scent infused flexible polymer.

4. The cannula of claim 1, wherein the scent infused polymer is a scent infused medical grade polymer.

5. The cannula of claim 1, wherein the scent infused polymer is a scent infused thermoplastic polymer.

6. The cannula of claim 1, wherein the scent infused polymer is a scent infused cellulosic polymer.

7. The cannula of claim 6, wherein the scent infused cellulosic polymer is a cellulose ester chosen from cellulose acetate butyrate, cellulose acetate propionate, cellulose acetate, or combination thereof.

8. The cannula of claim 1, wherein the tubular body and the pair of narine projections are formed from a scent infused polymer formulation, wherein the scent infused polymer formulation comprises:
the scent infused polymer, wherein the scent infused polymer comprises a scented compound and a polymer;
a plasticizer;
a stabilizer; and
a colorant.

9. The cannula of claim 8, wherein:
the polymer is a cellulosic polymer; and
the plasticizer is chosen from diethyl phthalate, triacetin, dioctyl adipate, diacetin, or combination thereof.

10. The cannula of claim 8, wherein the scent infused polymer formulation comprises:
greater than 50% by weight of the polymer;
from about 5% to about 40% by weight of the plasticizer;
from about 10% to about 30% by weight of the stabilizer;
less than 30% by weight of the scented compound; and
less than 10% by weight of the colorant.

11. The cannula of claim 1, wherein the scent which emits from the scent infused polymer is chosen from fruit, mint, chocolate, nut, flower, or combination thereof.

12. The cannula of claim 1, wherein the tubular body and the pair of narine projections are at least one of transparent or opaque.

13. The cannula of claim 1, wherein each of the tubular body and the pair of narine projections are a color that corresponds to the scent.

14. The cannula of claim 1, wherein the emission period is from about 1 hour to about 100 hours.

15. A method for providing a scent to a subject during nasal delivery of a fluid from a fluid source to the subject, the method comprising:
attaching a scent infused nasal cannula to the subject, wherein the scent infused nasal cannula comprises a nose piece and a pair of fluid supply tubes in open fluidic communication with the nose piece and in open fluid communication with the fluid source, and wherein the nose piece is formed from a scent infused polymer from which a scent emits for an emission period; and
delivering the fluid from the fluid source to the subject via the scent infused nasal cannula, wherein the fluid is delivered during at least a portion of the emission period of the scent infused polymer such that the scent is detectable therefrom.

16. The method of claim 15, wherein:
the fluid is oxygen; and
the oxygen is delivered to the subject at a rate of from about 1 to about 60 liters/minute.

17. The method of claim 15, wherein the scent infused polymer is a scent infused medical grade thermoplastic polymer.

18. The cannula of claim 15, wherein the scent infused polymer is a scent infused cellulosic polymer.

19. A method of fabricating a scent infused nasal cannula for nasal delivery of a fluid from a fluid source to a subject, the method comprising:
providing a scent infused nose piece comprising a tubular body and a pair of narine projections integral with and in fluidic communication with the tubular body, wherein the tubular body defines a first fluid inlet and a second fluid inlet, wherein the pair of narine projections define a first fluid outlet and a second fluid outlet, and wherein the scent infused nose piece is formed from a scent infused polymer from which a scent emits for an emission period; and
attaching a pair of fluid supply tubes to the first inlet of the tubular body, to the second inlet of the tubular body, and to the fluid source.

20. The method of claim 19, wherein the scent infused nose piece is formed from a scent infused polymer formulation, wherein the scent infused polymer formulation comprises:
the scent infused polymer, wherein the scent infused polymer comprises a scented compound and a cellulosic polymer chosen from cellulose acetate butyrate, cellulose acetate propionate, cellulose acetate, or combination thereof;
a plasticizer chosen from diethyl phthalate, triacetin, dioctyl adipate, diacetin, or combination thereof;
hexylene glycol; and
a colorant.
